(19) United States
(12) Patent Application Publication
(10) Pub. No.: US 2026/0069879 A1
(43) Pub. Date: Mar. 12, 2026

(54) Eyewear to be Used During Phototherapy

(71) Applicant: Shenzhen Kaiyan Medical Equipment Co. Ltd, Shenzhen (CN)

(72) Inventors: Alain Dijkstra, Amstelveen (NL); Chen Shoufeng, Shenzhen (CN); Guo Chunfang, Shenzhen (CN)

(21) Appl. No.: 18/827,572

(22) Filed: Sep. 6, 2024

(51) Int. Cl. A61N 5/06

(52) U.S. Cl. CPC A61N 5/06; A61N 2005/0626; A61N 2005/0642

(57) ABSTRACT

The present invention relates an eyewear (100) to be used during phototherapy. The eyewear (100) comprises a frame (10), a pair of lenses (11a, 11b) attached to the frame (10), a plurality of sensors (20) arranged within the frame (10), a processing unit (30), and a communication unit (40). The plurality of sensors (20) is configured to detect various parameters, including proximity, biometric data, temperature, distance, and light wavelength. The processing unit (30) is communicatively coupled with the plurality of sensors (20) and is adapted to process the data received from the plurality of sensors (20). The communication unit (40) is adapted to interact with a phototherapy device (200) to control at least one operational parameter of the phototherapy device (200) based on the processed data. The communication unit (40) is also adapted to transmit an alert to a user (A) through a speaker (29) arranged on the eyewear (100).

### TECHNICAL FIELD

The present invention relates to an eyewear. More specifically, the present invention relates to an eyewear to be used during phototherapy treatment.

### BACKGROUND

Phototherapy, also known as light therapy, is a versatile medical treatment used for a variety of conditions. One of its most common applications is for skin disorders such as psoriasis, eczema, and vitiligo. In these cases, ultraviolet (UV) light is utilized to slow down the abnormal growth of skin cells, reducing symptoms and improving the appearance of the skin. The treatment is usually administered in controlled doses to minimize potential side effects.

In neonatal care, phototherapy is an essential treatment for jaundice in newborns. Jaundice occurs when there is an excess of bilirubin in the blood, leading to yellowing of the skin and eyes. Phototherapy helps break down bilirubin into substances that the infant's body can easily excrete. This treatment is typically safe and effective, significantly reducing the risk of complications associated with high bilirubin levels.

Seasonal Affective Disorder (SAD) is another condition where phototherapy proves beneficial. SAD is a type of depression that occurs at a specific time of year, usually in the winter months when natural sunlight is limited. Light therapy boxes that mimic natural sunlight can help alleviate the symptoms of SAD by influencing the brain chemicals linked to mood and sleep. Regular sessions of light therapy can improve mood, energy levels, and overall well-being for those affected by this disorder.

Additionally, phototherapy is used to treat certain sleep disorders. By exposing patients to specific types of light at particular times of day, phototherapy can help regulate the body's internal clock, also known as the circadian rhythm. This regulation can be beneficial for individuals suffering from circadian rhythm disorders, such as delayed sleep phase disorder or shift work sleep disorder, leading to better sleep patterns and improved quality of life.

However, the light sources used in phototherapy can emit harmful wavelengths that pose significant risks to the eyes. Conventional protective eyewear used during phototherapy sessions often lacks the necessary features to make sure that the eyewear is worn by the user to provide safety to the eyes of the user.

The eyewear primarily focuses on blocking harmful UV rays. This existing protective eyewear lacks integrated sensors to monitor critical parameters such as light intensity, wavelength, and the distance between the user and the phototherapy device. Without real-time monitoring, it is difficult to ensure that the user is receiving the appropriate dosage of light therapy, which can compromise the treatment's effectiveness.

Current systems do not provide real-time feedback or alerts to the user about their positioning or the phototherapy device's operational status. This absence of feedback can result in improper usage, reducing the treatment's efficacy and potentially causing harm.

The present protective eyewear requires manual adjustments and does not interact with the phototherapy device. Users must rely on their judgment to position themselves correctly and ensure the proper functioning of the device, which is not always reliable.

Also, there is a lack of integration with biometric data to monitor the user's physiological responses. Without this data, the system cannot adjust the treatment parameters in real-time to suit the user's current physical condition, leading to suboptimal therapy.

Existing eyewear systems do not support wireless communication protocols, limiting their ability to interact with modern phototherapy devices and smart applications. This limitation restricts the ability to provide remote monitoring and control, which is increasingly important in modern medical treatments.

Therefore, there is a need for an eyewear to be used during phototherapy to overcome a few or all drawbacks of the existing technologies.

### STATEMENT OF THE INVENTION

An object of the present invention is to provide an eyewear to be used during phototherapy.

Another object of the present invention is to provide an eyewear to be used during phototherapy that can detect various parameters to ensure continuous monitoring of the treatment conditions and the user's status.

Yet another object of the present invention is to provide an eyewear to be used during phototherapy that provides real-time alerts and feedback to the user.

Another object of the present invention is to provide an eyewear to be used during phototherapy that detects the position of the eyewear during phototherapy sessions, ensuring the user's eyes are fully protected.

One more object of the present invention is to provide an eyewear to be used during phototherapy, which can detect improper positioning or unsafe conditions and provide a solution to prevent any potential harm to the user.

According to the present invention, there is provided an eyewear to be used during phototherapy. The eyewear may include a frame, a pair of lenses attached to the frame, a plurality of sensors, a processing unit, and a communication unit. Further, the eyewear may include an energy storage unit arranged within the frame, which provides energy to the processing unit, the communication unit, and the plurality of sensors, enabling the operation of the eyewear. The energy storage unit is a rechargeable battery.

The plurality of sensors may be configured to detect parameters including proximity, temperature, distance, and light wavelength. The plurality of sensors may include at least one proximity sensor, at least one temperature sensor, at least one distance sensor, and at least one light sensor.

The proximity sensor may be configured to determine a wearing status of the eyewear. The temperature sensor may be adapted to measure the ambient temperature around the user or the temperature of the user. Based on the measured temperature, the eyewear may adjust the intensity of the light of the phototherapy device to ensure user comfort and safety.

The distance sensor may be provided to measure the distance between the user and the phototherapy device. The light sensor may be provided to detect the wavelength and the intensity of light received by the eyewear.

The processing unit may be communicatively coupled with a plurality of sensors. The processing unit may be adapted to process data received from the plurality of sensors. Specifically, the processing unit may be adapted to analyze the wearing status of the eyewear based on the data received from the plurality of sensors. When the processing unit confirms the eyewear is worn by the user, the communication unit may communicate the wearing status with the phototherapy device to activate the phototherapy device.

Further, the processing unit may be configured to analyze the intensity of light coming from the phototherapy device, and the distance between the user and the phototherapy device to transmit the alert based on the analyzed light intensity and the distance between the user and the phototherapy device to the user using a speaker arranged within the frame. The alert may be a voice command to maintain the optimal distance between the user and the phototherapy device.

Further, the processing unit may be configured to analyze ambient environmental conditions such as the light intensity and the wavelength detected by the light sensor of the plurality of sensors and may adjust the light output of the phototherapy device to compensate for changes in external light conditions.

Further, the processing unit may be configured to analyze ambient environmental conditions, such as the light intensity and the wavelength detected by the light sensor, and may adjust the phototherapy device's light output to compensate for changes in external light conditions.

The communication unit may be interfaced with the processing unit to receive a processed data from the processing unit and is adapted to interact with a phototherapy device to control at least one operational parameter of the phototherapy device based on the processed data.

The operational parameters of the phototherapy device may include activation or deactivation of the phototherapy device. Further, the operational parameters may include the intensity of the light emitted by the phototherapy device, the duration of light exposure, the pulsation rate of the emitted light, a color spectrum of the emitted light, and an angular distribution of the emitted light to optimize therapeutic effectiveness.

Furthermore, the operational parameters may be the timing of light emission sequences to match circadian rhythms or treatment protocols and modulation of light based on real-time environmental feedback through the plurality of sensors. In another aspect, the operational parameters may include adaptive control of the light emission based on the user's biometric feedback, such as stress levels or heart rate, and synchronization of light therapy with other phototherapy devices that are in use by the user.

In a second aspect of the invention, the eyewear may include a frame, a pair of lenses attached to the frame, a plurality of sensors, a processing unit, and a communication unit. The plurality of sensors may be configured to detect parameters including biometric data, temperature, distance, and light wavelength. The plurality of sensors may include at least one biometric sensor, at least one temperature sensor, at least one distance sensor, and at least one light sensor.

The processing unit may be communicatively coupled with the plurality of sensors. The processing unit may be adapted to process data received from the plurality of sensors and communicate with the communication unit.

The communication unit may be arranged in the frame. The communication unit may interface with the processing unit to receive the processed data and is adapted to interact with a phototherapy device to control at least one operational parameter of the phototherapy device based on the processed data and/or the communication unit may be adapted to transmit an alert to a user based on the processed data.

The biometric sensor may be provided for measuring biometric data relating to the skin and/or eyes of a user to confirm that the eyewear is properly positioned over the user's eyes. The biometric sensor may be adapted to detect the presence of the user's eyes, wherein the detection of the presence of the user's eyes verifies that the eyewear is worn on the eyes and not on any other part of the body, thereafter activating the phototherapy device by communicating the wearing status with the phototherapy device through the communication unit.

In a third aspect of the invention, an eyewear may include a frame, a pair of lenses attached to the frame, a plurality of sensors, a processing unit, and a communication unit. The plurality of sensors may be configured to detect parameters including proximity biometric data, temperature, distance, and light wavelength.

The processing unit may be communicatively coupled with the plurality of sensors. The processing unit may be adapted to process data received from the plurality of sensors and communicate with the communication unit.

The communication unit may be arranged in the frame. The communication unit may interface with the processing unit to receive the processed data and may be adapted to interact with a phototherapy device to control at least one operational parameter of the phototherapy device based on the processed data and/or the communication unit may be adapted to transmit an alert to a user based on the processed data.

The communication unit may include a first body-communication unit that may be adapted to communicate a wearing status with the phototherapy device through physical contact with the user. Further, the phototherapy device may include a second body-communication unit adapted to receive the wearing status from the first body-communication unit to turn on the phototherapy device. The first body-communication unit and the second body-communication unit may communicate with each other through body-communication technology, which may use the conductive properties of the human body to transmit signals between the eyewear and the phototherapy device.

In a fourth aspect of the invention, the phototherapy device may be a handheld device for phototherapy treatment. The eyewear may be adapted to communicate with the phototherapy device which may be a handheld phototherapy device configured to receive the wearing status from the first body-communication unit via physical contact with the user. The first body-communication unit may communicate the wearing status continuously to keep the handheld phototherapy device ON until the handheld phototherapy device is held by the user and the eyewear is properly positioned over the user's eyes.

### BRIEF DESCRIPTION OF DRAWINGS

Other features and advantages of the invention will become apparent when reading the detailed description given below, purely by way of example and in a non-limitative manner, referring to the following figures:

FIG. 1 shows a perspective view of an eyewear to be used during phototherapy in accordance with the present invention;

FIG. 2a shows a perspective view of the eyewear to be used during phototherapy of FIG. 1;

FIG. 2b shows a perspective view of an eyewear to be used during phototherapy in accordance with one of the embodiments of the present invention;

FIG. 3 shows a side view of the eyewear worn by a user in front of a phototherapy device;

FIG. 4 shows a schematic view of the eyewear communicating with the phototherapy device;

FIG. 5a-5d shows a side view illustrating the positions of the user in accordance with the present invention;

FIG. 6a-6b shows a perspective view of an eyewear to be used during phototherapy in accordance with a second embodiment of the present invention;

FIG. 7 shows a schematic view of an eyewear to be used during phototherapy in accordance with a third embodiment of the present invention;

FIG. 8a-8b shows a side view showing the positions of a user during phototherapy in accordance with the third embodiment of the present invention; and

FIG. 9a-9b shows a perspective view of a handheld device used during phototherapy in accordance with a fourth embodiment of the present invention.

### DETAILED DESCRIPTION

An embodiment of this invention, illustrating its features, will now be described in detail. The words “comprising,” “having,” “containing,” and “including,” and other forms thereof, are intended to be equivalent in meaning and be open-ended in that an item or items following any one of these words is not meant to be an exhaustive listing of such item or items, or meant to be limited to only the listed item or items.

The present invention provides an eyewear to be used during phototherapy. The eyewear is adapted to monitor various parameters related to the user's environment and physiological state. The monitoring of the various parameters allows to provide real-time adjustments and alerts, ensuring optimal conditions for phototherapy. Further, the eyewear is adapted for seamless interaction between the eyewear and the phototherapy device, facilitating precise control over treatment parameters.

The terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item.

The disclosed embodiments are merely exemplary of the invention, which may be embodied in various forms.

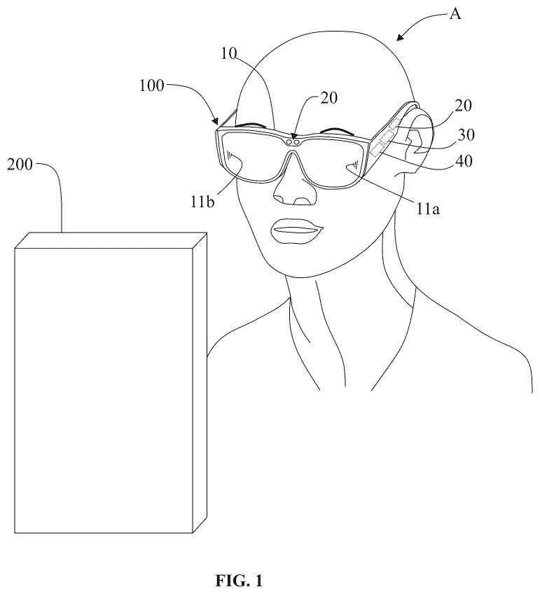

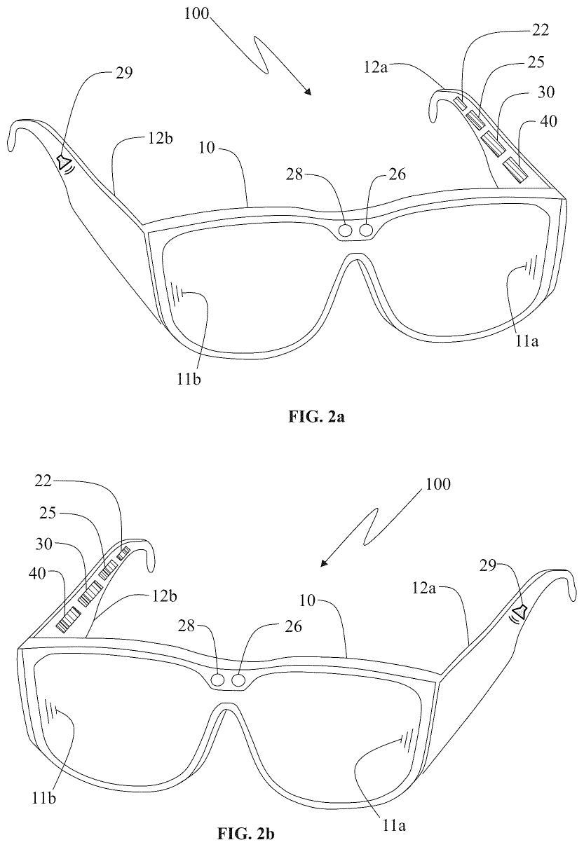

Referring now to FIGS. 1, 2a, and 2b, an eyewear (100) to be used during phototherapy in accordance with the present invention is illustrated. Specifically, the eyewear (100) includes a frame (10), a pair of lenses (11a, 11b) attached to the frame (10), a plurality of sensors (20) arranged in the frame (10), a processing unit (30) communicatively coupled with the plurality of sensors (20), and a communication unit (40) interfaced with the processing unit (30) to receive a processed data from the processing unit (30).

Further, the eyewear (100) includes an energy storage unit (90) arranged within the frame (10), which provides energy to the processing unit (30), the communication unit (40), and the plurality of sensors (20), enabling the operation of the eyewear (100) in the form of DC current. In the present embodiment, the energy storage unit (90) is a rechargeable battery.

The frame (10) of the eyewear (100) is adapted to receive the pair of lenses (11a, 11b) therein. The frame (10) is adapted to be placed over the nose of a user (A) and the pair of lenses (11a, 11b) are positioned adjacent to each other on the frame (10) using grooves or clips (not shown). The pair of lenses (11a, 11b) are adapted to cover the eyes of the user (A) tightly, preventing radiation from entering the eyes. In the present embodiment, the eyewear (100) is a spectacle. The eyewear (100) can be configured like spectacles used for swimming.

Further, the eyewear (100) includes a pair of temple arms (12a, 12b) extending from the frame (10), which can be mounted on the ears of the user (A). Specifically, a proximal end of each temple arm (12a, 12b) is pivotally connected/hingedly connected to the frame (10) at respective pivot points/hinge points near opposite lateral ends of the frame (10). The temple arms (12a, 12b) are configured to extend rearwardly from the frame (10) when in use, to support the eyewear (100) on a wearer's ears. The pivotal connection allows each temple arm (12a, 12b) to rotate between an open position for wearing and a folded position for storage. In the folded position, the pair of temple arms (12a, 12b) are folded over each other to configure the non-use position for portability. In the open position, the pair of temple arms (12a, 12b) are unfolded using the hinge elements to configure the use position. In this position, the eyewear (100) can be worn by the user (A).

Further, the plurality of sensors (20) is configured on the pair of temple arms (12a, 12b) to detect parameters such as proximity, temperature, etc. Specifically, the plurality of sensors (20) is configured on an inner side of the temple arms (12a, 12b). Further, the plurality of sensors (20) is configured on the frame (10) to detect parameters such as distance, light wavelength, etc. It may be obvious for a person skilled in the art to configure the plurality of sensors (20) on any other portion of the frame (10) or the temple arms (12a, 12b) of the eyewear (100) according to the required detecting parameter.

In the present embodiment, the plurality of sensors (20) includes at least one proximity sensor (22), at least one temperature sensor (25), at least one distance sensor (26), and at least one light sensor (28).

In the present embodiment, the proximity sensor (22) is arranged on one of the temple arms (12a, 12b) and adapted to detect the proximity between the temple arm (12a, 12b) and the user's head to ensure that the eyewear (100) is worn by the user (A). Specifically, the proximity sensor (22) is arranged in the left temple arm (12a) (shown in FIG. 2a). It may be obvious for a person skilled in the art to arrange the proximity sensor (22) in the right temple arm (12b) (shown in FIG. 2b).

More specifically, the proximity sensor (22) is arranged on the inner side of the temple arm (12a) in such a way that the proximity sensor (22) can detect the proximity between the temple arm (12a) and the user's head. It may be obvious for a person skilled in the art to arrange the proximity sensor (22) on any part of the eyewear (100) to detect the wearing status of the eyewear (100). In the present embodiment, the proximity sensor (22) is an infrared (IR) proximity sensor. It may be obvious for a person skilled in the art to use capacitive proximity sensors, inductive proximity sensors, ultrasonic proximity sensors, photoelectric proximity sensors, magnetic proximity sensors, laser proximity sensors, radar proximity sensors, or any other sensor as the proximity sensor (22).

The wearing status of the eyewear (100) is categorized into two distinct states: the first status and the second status. In the first status, the eyewear (100) is properly worn by the user (A), which means the eyewear (100) is positioned correctly on the user's eyes and in the second status, the eyewear (100) is not worn by the user (A), indicating that the eyewear (100) is not on the user's eye or is improperly worn.

The first status and the second status are detected by the plurality of sensors (20) arranged within the eyewear (100) by using data such as proximity, temperature, distance, and light wavelength which are continuously monitored. This data is used to control a phototherapy device (200) for phototherapy treatment and provide safety to the user (A) during the phototherapy treatment. In the first status, data such as proximity, temperature, distance, and light wavelength are continuously monitored by the plurality of sensors (20). In the second status, when the proximity sensor (22) detects that the eyewear (100) is not worn, the eyewear (100) is turned OFF and no further data is received from the plurality of sensors (20) until the proximity sensor (22) detects that the eyewear (100) is worn.

Further, the plurality of sensors (20) simultaneously detects parameters, such as proximity, and the user's temperature data for analyzing the wearing status. The plurality of sensors (20) detects whether the eyewear (100) is being worn or not, enabling the operation of the phototherapy device (200) based on the detected status. The processing unit (30) is adapted to process the data received from the plurality of sensors (20) to detect the wearing status of the eyewear (100).

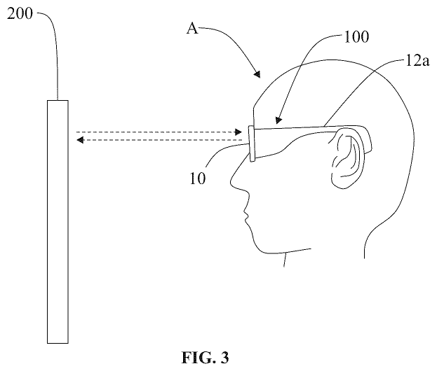

Preferably, if the proximity sensor (22) detects that the eyewear (100) is worn by the user (A) (the first status), the processing unit (30) communicates with the phototherapy device (200) through the communication unit (40) to activate the phototherapy device (200). The processing unit (30) is adapted to continuously send the wearing status (shown in FIG. 3) to the phototherapy device (200) that the eyewear (100) is worn by the user (A). Once the proximity sensor (22) detects that the eyewear (100) is being removed from the head of the user (A) (the second status), the processing unit (30) communicates the wearing status that the eyewear (100) is removed from the head of the user (A) (the second status), through the communication unit (40) to turn OFF the phototherapy device (200).

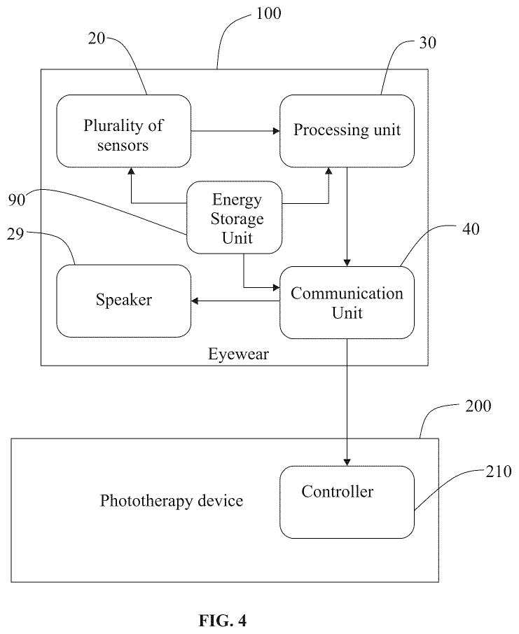

Specifically, the phototherapy device (200) has a controller (210) to receive the wearing status from the processing unit (30) through the communication unit (40) of the eyewear (100) (shown in FIG. 4).

Further, the temperature sensor (25) of the eyewear (100) is arranged on the frame (10) or the temple arm (12a, 12b) of the eyewear (100) to measure the ambient temperature around the user (A) or the temperature of the user (A). Based on the measured temperature, the processing unit (30) of the eyewear (100) is adapted to communicate with the phototherapy device (200) to adjust the intensity of the light of the phototherapy device (200) to ensure the comfort and safety of the user (A). It is obvious for a person skilled in the art to use the temperature sensor (25) which may be a thermistor, infrared (IR) sensor, semiconductor sensor, or any other sensor that is adapted to be arranged on the eyewear (100) and is suitable for detecting the user's temperature or the ambient temperature.

Further, the distance sensor (26) is arranged on the frame (10) of the eyewear (100) directing outwards to measure the distance between the user (A) and the phototherapy device (200). In the present embodiment, the distance sensor (26) is an ultrasonic sensor. It may be obvious for a person skilled in the art to use infrared (IR) sensors, laser rangefinders (LIDAR), time-of-flight (TOF) sensors, radar sensors, capacitive proximity sensors, inductive proximity sensors, photoelectric sensors, magnetic proximity sensors, optical distance sensors or any other sensor suitable for measuring the distance between two objects as the distance sensor (26).

The distance sensor (26) is adapted to continuously monitor and measure the distance between the eyewear (100) and the phototherapy device (200), ensuring that the user (A) wearing the eyewear (100) is within the optimal range for effective treatment. The distance sensor (26) is arranged to provide real-time feedback and adjustments to maintain the correct therapeutic distance.

Further, the light sensor (28) is arranged on the eyewear (100) to detect the wavelength and the intensity of light received by the eyewear (100). The light sensor (28) is arranged preferably on the frame (10) directing outwards to detect the intensity of the light coming from the phototherapy device (200). Further, the light sensor (28) is adapted to detect the wavelength of the light and the intensity of the light and communicate with the processing unit (30) to send the detected data. In the present embodiment, the light sensor (28) is a photodiode. It may be obvious for a person skilled in the art to use any other sensor as the light sensor (28) to detect the wavelength and the intensity of the light.

The plurality of sensors (20) continuously detects parameters like proximity, temperature, distance, and light wavelength and communicates with the processing unit (30) of the eyewear (100).

The processing unit (30) is arranged within the frame (10) or the temple arms (12a, 12b) of the eyewear (100). The processing unit (30) is communicatively coupled with the plurality of sensors (20) and is provided to process the data received from the plurality of sensors (20). The processing unit (30) processes the data received from the plurality of sensors (20) to operate the phototherapy device (200), providing safety to the user (A) while using the phototherapy device (200).

Moreover, the processed data of the plurality of sensors (20) is communicated with the communication unit (40) in the first status. The communication unit (40) is arranged within the frame (10) or the temple arms (12a, 12b) of the eyewear (100) and is interfaced with the processing unit (30) to receive the processed data. The communication unit (40) communicates with the phototherapy device (200) through a wireless communication protocol such as Bluetooth or Wi-Fi, enabling the eyewear (100) to interact with the phototherapy device (200) to control the phototherapy device (200) or a smartphone or tablet app that controls the phototherapy device (200).

The processing unit (30) is adapted to analyze the wearing status of the eyewear (100) based on the data received from the plurality of sensors (20), specifically the data from the proximity sensor (22). The processing unit (30) processes the data received from the proximity sensor (22) in several steps. First, the processing unit (30) analyzes the data received from the proximity sensor (22) to determine the distance between the eyewear (100) and the user's skin/head. The processing unit (30) confirms whether the eyewear (100) is being worn on the user's head or not. If the processing unit (30) confirms that the eyewear (100) is worn by the user (A), the communication unit (40) communicates the wearing status with the phototherapy device (200) particularly with the controller (210) of the phototherapy device (200) to activate the phototherapy device (200).

Furthermore, the processing unit (30) processes the data received from the plurality of sensors (20) to detect the user's position relative to the phototherapy device (200). Preferably, the distance sensor (26) detects the user's position relative to the phototherapy device (200) to check whether the user (A) is within the optimal range and positioned within the effective angle of the phototherapy device (200), and the light sensor (28) detects the intensity of the light coming from the phototherapy device (200). The distance sensor (26) and the light sensor (28) are adapted to check whether the user (A) is positioned for the optimal use of the phototherapy light or not.

The eyewear (100) further includes a speaker (29) to transmit an alert of the optimal use of the phototherapy light and the position of the user (A) within the effective angle. The speaker (29) is arranged on the temple arms (12a, 12b) of the eyewear (100) to alert the user (A) through a voice command or vibrations. Specifically, the processing unit (30) is configured to analyze the intensity of light emitting from the phototherapy device (200) and transmit the alert based on the analyzed light intensity to the communication unit (40). The communication unit (40) is interfaced with the processing unit (30) and the speaker (29) in such a way that upon receiving the alert from the processing unit (30), the communication unit (40) transmits the alert through the speaker (29).

Specifically, the communication unit (40) communicates the voice command to maintain the optimal distance between the user (A) and the phototherapy device (200), or the position of the user (A) according to the effective angle of the phototherapy device (200).

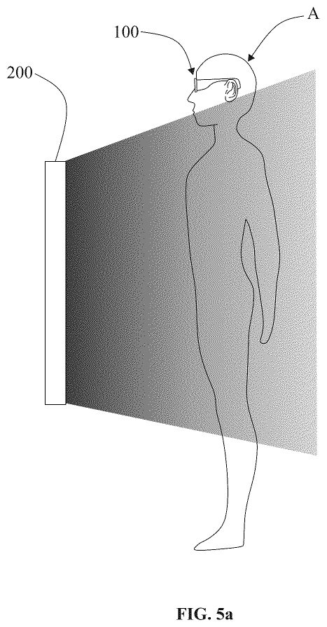

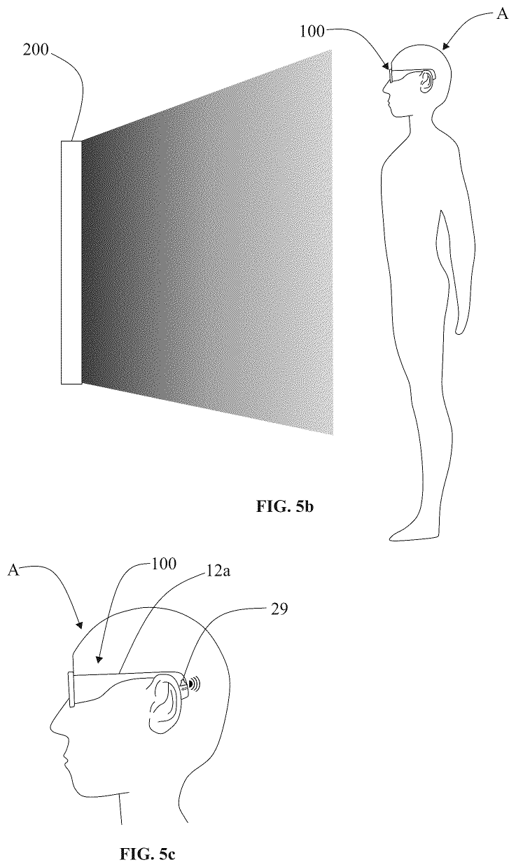

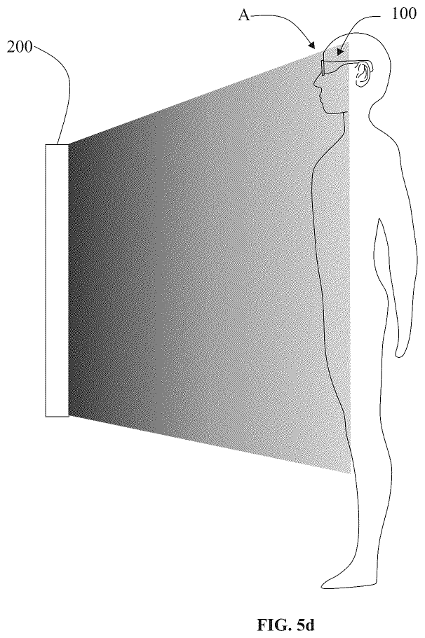

Further, in an exemplary embodiment as shown in FIG. 5a-5d, the communication unit (40) is adapted to communicate the data detected by the distance sensor (26) and the light sensor (28) simultaneously. The distance sensor (26) detects the position of the user (A) with respect to the phototherapy device (200) and the light sensor (28) detects the intensity of the light coming from the phototherapy device (200) and communicates with the processing unit (30). The processing unit (30) processes the data received from the distance sensor (26) and the light sensor (28). If the user (A) is not positioned within the optimal distance (FIG. 5b) or is positioned too close to the phototherapy device (200) (shown in FIG. 5a), the processing unit (30) transmits the voice command through the communication unit (40) for the user (A) to position himself within the optimal distance (FIG. 5d) for effective phototherapy treatment.

If the user (A) is positioned away from the phototherapy device (200) (FIG. 5b) and the light from the phototherapy device (200) is not reaching the user (A), then the communication unit (40) transmits the command through the speaker (29) that the user (A) needs to move forward towards the phototherapy device (200) to receive the light for the effective phototherapy treatment. If the user (A) is positioned too close to the phototherapy device (200) (FIG. 5a) and the excessive light from the phototherapy device (200) reaches to the user (A), then the communication unit (40) transmits the command through the speaker (29) that the user (A) needs to move away from the phototherapy device (200).

Furthermore, the communication unit (40) is adapted to interact with the phototherapy device (200) to control at least one operational parameter of the phototherapy device (200) based on the processed data. The operational parameters of the phototherapy device (200) include an activation or deactivation of the phototherapy device (200), an intensity of the light, a duration of light, a pulsation rate, a color spectrum, angular distribution of the emitted light, timing of light emission sequences, modulation of light, adaptive control of the light, and synchronization of light therapy.

The activation or deactivation of the phototherapy device (200) means controlling the working state of the phototherapy device (200) to turn it ON or OFF as needed. Depending on the data received from the proximity sensor (22), the processing unit (30) communicates with the phototherapy device (200) using the communication unit (40) to turn ON or OFF the phototherapy device (200).

The intensity of the light emitted by the phototherapy device (200) is an adjustment of the brightness level of the light emitted by the phototherapy device (200). Specifically, the light sensor (28) of the plurality of sensors (20) detects ambient environmental conditions and communicates with the processing unit (30). The processing unit (30) is configured to analyze ambient environmental conditions, such as the light intensity and the wavelength detected by the light sensor (28) to control at least one operational parameter of the phototherapy device (200), such as the intensity of the light emitted by the phototherapy device (200), the duration of light exposure, the pulsation rate of the emitted light, the color spectrum of the emitted light and modulation of light. Specifically, the processing unit (30) transmits the analyzed data to the communication unit (40) which further communicates with the phototherapy device (200). Specifically, the communication unit (40) communicates with the controller (210) of the phototherapy device (200) to adjust the light output of the phototherapy device (200) to compensate for changes in external light conditions.

Similarly, the temperature sensor (25) measures the ambient temperature around the user (A) or the temperature of the user, and based on the measured temperature, the eyewear (100) adjusts the intensity of the light of the phototherapy device (200) to ensure user (A) comfort and safety. More specifically, the measured temperature is analyzed by the processing unit (30), and based on the analyzed temperature, the processing unit (30) communicates with the controller (210) of the phototherapy device (200) through the communication unit (40) to adjust the intensity of the light of the phototherapy device (200).

Further, the duration of light exposure control means setting the length of time, the user (A) is exposed to the phototherapy light. For controlling the duration of the light exposure, the processing unit (30) of the eyewear (100) communicates with the controller (210) to adjust the time of the phototherapy session to control how long the user (A) is exposed to the therapeutic light to ensure that the treatment duration is optimal preventing overexposure which could lead to adverse effects and ensuring sufficient exposure for the effectiveness of the phototherapy treatment.

The pulsation rate of the emitted light means modulating the frequency at which the light pulses or blinks. The communication unit (40) is adapted to communicate with the controller (210) to vary the pulsation or blinking rate of the light. Specifically, the processing unit (30) communicates the pulsation rate with the phototherapy device (200) through the communication unit (40) to control the pulsation rate of the emitted light from the phototherapy device (200). The processing unit (30) is adapted to analyze the data received from the plurality of sensors (20) such as the light sensor (28) and the temperature sensor (25).

Further, the color spectrum of the emitted light is adjusted by altering the range of wavelengths or colors of the emitted light. The communication unit (40) of the eyewear (100) is adapted to communicate the data received from the plurality sensors (20) with the phototherapy device (200) to adjust the color spectrum of the light emitted.

To control the angular distribution of the emitted light, the direction and spread of the light are changed to target specific areas to optimize therapeutic effectiveness. Specifically, the communication unit (40) of the eyewear (100) is adapted to communicate with the phototherapy device (200) to modify the angle at which light is emitted from the phototherapy device (200) to ensure the phototherapy device (200) targets the specific area of the body. Preferably, the processing unit (30) receives the data from the light sensor (28) and the distance sensor (26) to analyze the angular distribution of the emitted light and control the same according to the user's position detected by the distance sensor (26) and the intensity of the light detected by the light sensor (28).

Furthermore, the timing of the light emission sequence is scheduled to match circadian rhythms or treatment protocols. The processing unit (30) of the eyewear (100) is adapted to schedule light emission to align with the user's natural circadian rhythms or specific treatment protocols to enhance the effectiveness of treatments, particularly for conditions related to sleep and mood disorders.

The modulation of light is based on real-time environmental feedback through the plurality of sensors (20) to adjust the light output. Specifically, the processing unit (30) of the eyewear (100) facilitates the adjustment of the light output from the phototherapy device (200) in response to changes in the surrounding environment, such as ambient light levels. The changes in the surrounding environment are detected by the light sensor (28) and transmitted to the processing unit (30). In another embodiment, the changes in the surrounding environment are detected by any other sensors that are suitable to check the changes in light.

The adaptive control of the light emission is based on the user's physiological data such as stress levels or heart rate. The eyewear is adapted to turn the phototherapy device (200) ON or OFF automatically based on the wearing status or specific treatment schedules to ensure that the phototherapy device (200) operates only when necessary and according to the detected physiological data such as stress levels or heart rate.

Further, the synchronization of light therapy with other phototherapy devices to coordinate the light therapy sessions with other phototherapy devices being used by the user (A). The eyewear (100) is adapted to coordinate with other phototherapy devices specifically, the communication unit (40) is adapted to coordinate with other phototherapy devices that are being used simultaneously by the user (A) to control them according to the data detected by the plurality of the sensors.

The eyewear (100) is adapted to communicate the wearing status with the phototherapy device (200) to detect parameters for continuous monitoring of the phototherapy treatment and the user's status. Further, the eyewear (100) provides real-time alerts and feedback to the user (A) using the speaker (29) to ensure the position of the user (A) within the optimal range. The eyewear (100) is adapted to detect the position of the eyewear (100) during phototherapy sessions, ensuring the user's eyes are fully protected.

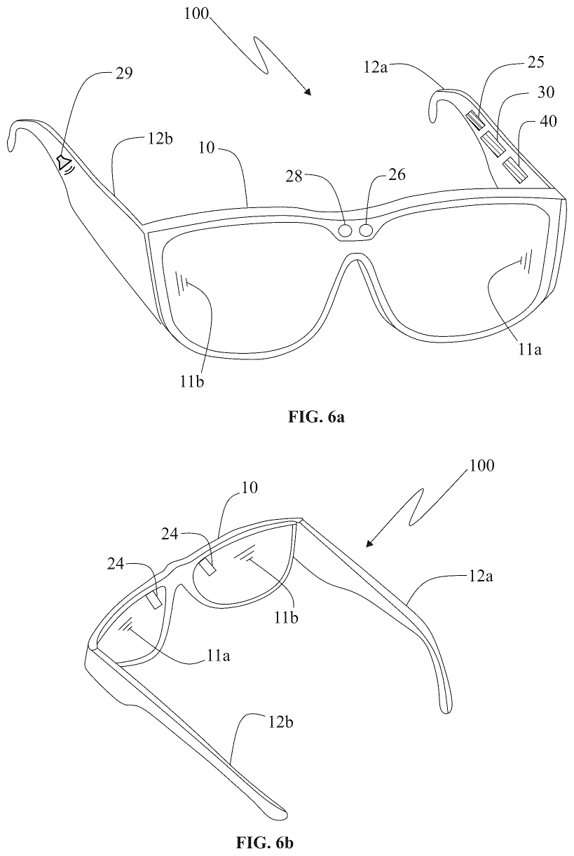

In the second embodiment (as shown in FIG. 6a-6b) of the invention, the eyewear (100) includes a frame (10), a pair of lenses (11a, 11b) attached to the frame (10), a plurality of sensors (20) arranged in the frame (10), a processing unit (30) communicatively coupled with the plurality of sensors (20), and a communication unit (40) interfaced with the processing unit (30) to receive the processed data from the processing unit (30).

The frame (10) of the eyewear (100) is adapted to receive the pair of lenses (11a, 11b) therein. Further, the eyewear (100) has a pair of temple arms (12a, 12b) extending from the frame (10), which can be mounted on the ears of the user (A). Further, the plurality of sensors (20) is configured on the frame (10) to detect parameters such as biometric data, temperature, distance, and light wavelength. The plurality of sensors (20) continuously detects parameters like proximity, temperature, distance, and light wavelength and communicates with the processing unit (30) of the eyewear (100).

The processing unit (30) is communicatively coupled with the plurality of sensors (20). The processing unit (30) is adapted to process data received from the plurality of sensors (20) and communicate with the communication device (40). The communication unit (40) of the eyewear (100) is interfaced with the processing unit (30) to receive the processed data and is adapted to interact with the phototherapy device (200) to control at least one operational parameter of the phototherapy device (200) based on the processed data.

The plurality of sensors (20) includes at least one biometric sensor (24), at least one temperature sensor (25), at least one distance sensor (26), and at least one light sensor (28). The processing unit (30) is adapted to receive the data from the plurality of sensors (20) to detect the wearing status of the eyewear (100).

The biometric sensor (24) is positioned on the frame (10) of the eyewear (100) exposed (shown in FIG. 6b) to detect the presence of the user's skin and/or eyes preferably to detect the liveness of the human eye and measure the biometric data related to the user's skin and/or eyes. In this embodiment (as shown in FIG. 6b), the biometric sensor (24) is arranged on the frame (10) extending parallel to the pair of lenses (11a, 11b) to detect the user's eye accurately. Preferably, two biometric sensors (24) are arranged on the frame (10) to detect the biometric data of each eye. The biometric sensor (24) is provided to detect the presence of the eye ensuring that the eyewear (100) is properly positioned over the user's eyes.

The biometric sensor (24) is adapted to measure the biometric data such as eye characteristics of user's eyes like pupil size, blink rate, and movements of the eyeball.

Specifically, in the second embodiment, for the detection of the eyes, the biometric sensor (24) is an Infrared (IR) eye-tracking sensor. The IR eye tracking sensor uses infrared light-emitting diodes (LEDs) to illuminate the eye. The infrared light reflects off the different parts of the eye, including the cornea and the retina. The reflected light is captured by a camera of the IR eye tracking sensor positioned around the eye. Specifically, the IR eye-tracking sensor is arranged on the inner side of the frame (10) and directed towards the eyes of the user (A). The IR eye tracking sensor is arranged on the frame (10) in such a way that the eyes of the user (A) are visible to the camera to capture the pupil size, blink rate, and movements of the eyeball. It may be obvious for a person skilled in the art to use any other type of sensor such as electrooculography (EOG) sensors, photoplethysmography (PPG) sensors, optical coherence tomography (OCT) sensors, or any other sensor for detecting biometric parameters of the eye.

The biometric sensor (24) detects features like eye shape, pupil size, blink rate, or movement of the eyeball inside the eyelids to ensure the eyewear (100) is correctly aligned over the eyes.

Further, the biometric sensor (24) is adapted to detect the liveness of the iris of the user (A). The detection of the liveliness of the iris verifies that the eyewear (100) is worn on the eyes and not on any other part of the body. The detection of the liveness of the iris works by analyzing patterns in the iris, pupil response to light, and other characteristics unique to a living eye. In another embodiment, the biometric sensor (24) is adapted to detect the stress level or heart rate of the user (A).

By confirming the detection of the eyes, eyeball movement, or the presence of an iris, the processing unit (30) analyzes the data received from the biometric sensor (24) to differentiate between the eyewear (100) being worn on the eyes or being placed on other parts of the body, such as the forehead or around the neck.

Upon detection of the eyewear (100) being worn by the user (A), the processing unit (30) communicates with the phototherapy device (200) through the communication unit (40) to activate the phototherapy device (200). The processing unit (30) is adapted to continuously send the wearing status to the phototherapy device (200) that the eyewear (100) is worn by the user (A) on the eyes. Once the biometric sensor (24) detects that the eyewear (100) is being removed from the user's eyes (the second status), the processing unit (30) communicates the wearing status that the eyewear (100) is removed from the eyes of the user (A) (the second status), through the communication unit (40) to turn OFF the phototherapy device (200).

The plurality of sensors (20) continuously detects parameters like biometric data, temperature, distance, and light wavelength and communicates with the processing unit (30) of the eyewear (100). The plurality of sensors (20) provide data about whether the eyewear (100) is properly positioned on the user's eyes during the phototherapy treatment.

Further, the processing unit (30) processes the data received from the plurality of sensors (20) to detect the user's position relative to the phototherapy device (200). Preferably, the distance sensor (26) detects the user's position relative to the phototherapy device (200) to check whether the user (A) is within the optimal range and positioned within the effective angle of the phototherapy device (200).

Furthermore, the light sensor (28) of the plurality of sensors (20) is configured to detect the light intensity and the wavelength to analyze ambient environmental conditions, for controlling at least one operational parameter of the phototherapy device (200).

Similarly, the temperature sensor (25) measures the ambient temperature around the user (A), and based on the measured temperature, the processing unit (30) eyewear (100) adjusts the intensity of the light of the phototherapy device (200) to ensure comfort and safety to the user (A).

In an embodiment, the eyewear (100) is adapted to communicate real-time biometric data with the phototherapy device (200) to modify light emission based on real-time biometric data of the user (A) such as stress levels or heart rate.

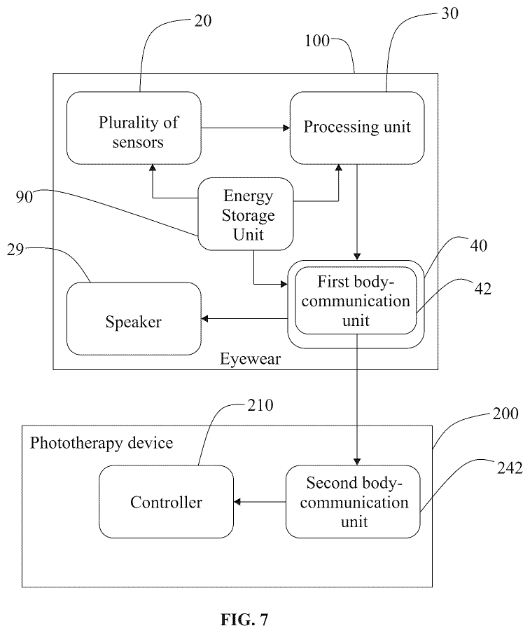

Referring now to FIG. 7, 8a-8b, in a third embodiment, an eyewear (100) to be used during phototherapy in accordance with the third embodiment of the invention is provided. The eyewear (100) includes a frame (10), a pair of lenses (11a, 11b) attached to the frame (10), a plurality of sensors (20) arranged in the frame (10), a processing unit (30) communicatively coupled with the plurality of sensors (20), and a communication unit (40) interfaced with the processing unit (30) to receive processed data from the processing unit (30).

The frame (10) of the eyewear (100) is adapted to receive the pair of lenses (11a, 11b) therein. Further, the eyewear (100) has a pair of temple arms (12a, 12b) extending from the frame (10), which can be mounted on the ears of the user (A). Further, the plurality of sensors (20) is configured on the frame (10) to detect parameters such as proximity, biometric data, temperature, distance, and light wavelength. The plurality of sensors (20) is adapted to communicate with the processing unit (30) to send detected parameters.

Furthermore, the processing unit (30) is communicatively coupled with the plurality of sensors (20). The processing unit (30) is adapted to process data received from the plurality of sensors (20) and communicate with the communication device (40). The communication unit (40) of the eyewear (100) is interfaced with the processing unit (30) to receive processed data and is adapted to interact with the phototherapy device (200) to control at least one operational parameter of the phototherapy device (200) based on the processed data.

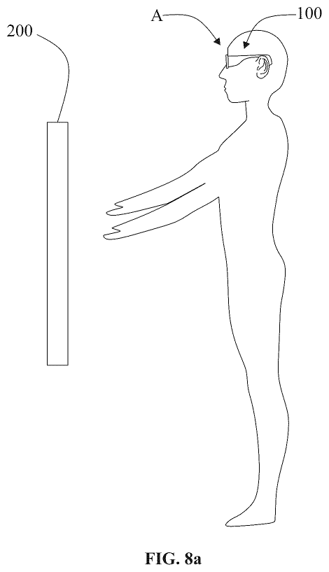

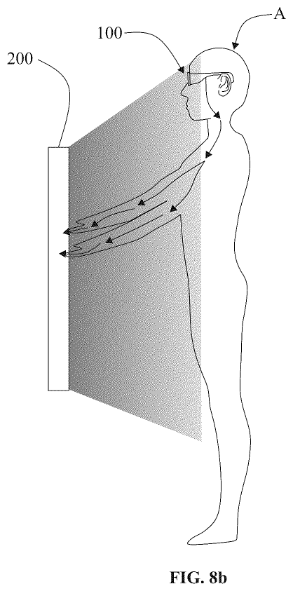

In the third embodiment, the communication unit (40) of the eyewear (100) includes a first body-communication unit (42), and the phototherapy device (200) includes a second body-communication unit (242). The first body-communication unit (42) is adapted to communicate the wearing status with the phototherapy device (200) through physical contact with the user (A). Specifically, when the user (A) touches both the eyewear (100) and the phototherapy device (200), the first body-communication unit (42) communicates the wearing status via the user's body (as shown in FIG. 8b). The second body-communication unit (242) of the phototherapy device (200) is adapted to receive the wearing status from the first body-communication unit (42) to turn ON or OFF the phototherapy device (200).

Specifically, if the second body-communication unit (242) receives the wearing status that the eyewear (100) is properly positioned over the eyes, the controller (210) of the phototherapy device (200) turns ON the phototherapy device (200), otherwise, the phototherapy device (200) is turned OFF (as shown in FIG. 8a).

The communication between the eyewear (100) and the phototherapy device (200) is facilitated by body-communication technology, which uses the conductive properties of the human body to transmit signals between the eyewear (100) and the phototherapy device (200). The first body-communication unit (42) and the second body-communication unit (242) are adapted to use the user's body as a transmission medium, utilizing the natural conductivity of the skin to send the wearing status.

By way of non-limiting example, the eyewear (100) and the phototherapy device (200) use a Non-radiative Wire-like wireless (Wi-R) technology. The Wi-R is a non-radiative near-field communication technology that uses Electro-Quasistatic (EQS) Fields for communication. The Wi-R technology creates a body area network (BAN) to connect the eyewear (100) with the phototherapy device (200) through the body of the user (A) for establishing communication between the eyewear (100) and the phototherapy device (200).

It may be obvious for a person skilled in the art to use various body-communication technologies to transmit the wearing status from the eyewear (100) to the phototherapy device (200).

When the user (A) touches the phototherapy device (200), the second body-communication unit (242) detects the wearing status transmitted from the first body-communication unit (42), ensuring that the phototherapy device (200) only activates when the user (A) is wearing the eyewear (100) and is in the correct position to receive the phototherapy treatment. If the eyewear (100) is not worn or is not in the correct position, the phototherapy device (200) is deactivated (shown in FIG. 8a) and is not turned ON until the phototherapy device (200) processing unit (30) detects that the eyewear (100) is in the first status ensuring that the eyewear (100) is properly positioned over the eyes.

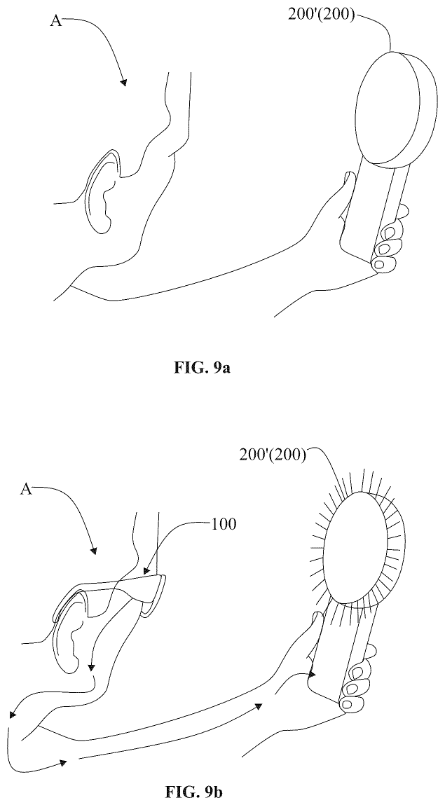

Referring to FIG. 9a-9b, in a fourth embodiment, the phototherapy device (200) is a handheld device (200′) that is held in the hand of the user (A) and is moved by the user (A) near the skin to receive the emitted light on the skin. In preferred use, the handheld device (200′) is used for the treatment of the face, hence confirming the eyewear (100) worn by the user (A) is essential for ensuring the safety of the user's eyes. The processing unit (30) of the eyewear (100) detects the wearing status using the plurality of sensors (20) and sends the wearing status signal to the communication unit (40). The communication unit (40) has the first body-communication unit (42) to transmit the wearing status to the second body-communication unit (242) of the phototherapy device (200). Upon receiving the wearing status, that is the eyewear (100) is properly positioned over the eyes, the second body-communication unit (242) sends the signal to the controller (210) to activate the handheld device (200′) (shown in FIG. 9b). The wearing status of the eyewear (100) is continuously monitored using the plurality of sensors (20) to check whether the eyewear (100) is worn by the user (A) and is properly positioned over the eyes during phototherapy treatment.

In one more embodiment of the present invention, the phototherapy device (200) is a wearable phototherapy device (200) such as a face patch, or eyepatch. The wearable phototherapy device (200) is adapted to stick to the skin of the user (A) to effectively emit light on the skin of the user (A). Further, the eyewear (100) includes a power source that is used to power the wearable phototherapy device (200) through the first body-communication unit (42) of the eyewear (100) and the second body-communication unit (242) of the wearable phototherapy device (200).

Therefore, the present invention has the advantage of providing an eyewear (100) to be used during phototherapy. The eyewear (100) is adapted to communicate the wearing status with the phototherapy device (200) to detect parameters to ensure continuous monitoring of the treatment conditions and the user's status. Further, the eyewear (100) provides real-time alerts and feedback to the user (A) using the speaker (29). The eyewear (100) is adapted to detect the position of the eyewear (100) during phototherapy sessions, ensuring the user's eyes are fully protected. Furthermore, the eyewear (100) is adapted to detect improper positioning or unsafe conditions and provide a solution to prevent any potential harm to the user (A).

The foregoing descriptions of specific embodiments of the present invention have been presented for purposes of illustration and description. They are not intended to be exhaustive or to limit the present invention to the precise forms disclosed, and obviously, many modifications and variations are possible in light of the above teaching. The embodiments were chosen and described in order to explain the principles of the present invention best and its practical application, thereby enabling others skilled in the art to best utilize the present invention and various embodiments with various modifications as are suited to the particular use contemplated. It is understood that various omission and substitutions of equivalents are contemplated as circumstance may suggest or render expedient, but such are intended to cover the application or implementation without departing from the scope of the claims of the present invention.

1. An eyewear (100) to be used during phototherapy, the eyewear (100) comprising:
a frame (10);
a pair of lenses (11a, 11b) attached to the frame (10);
a plurality of sensors (20) arranged in the frame (10), the plurality of sensors (20) configured to detect parameters including proximity, biometric data, temperature, distance, and light wavelength;
a processing unit (30) communicatively coupled with the plurality of sensors (20), the processing unit (30) is adapted to process data received from the plurality of sensors (20), and
a communication unit (40) interfaced with the processing unit (30) to receive a processed data from the processing unit (30) and is adapted to interact with a phototherapy device (200) to control at least one operational parameter of the phototherapy device (200) based on the processed data and/or is adapted to transmit an alert to a user (A) based on the processed data.

2. The eyewear (100) as claimed in claim 1, wherein the plurality of sensors (20) comprises:
at least one proximity sensor (22) configured to determine a wearing status of the eyewear (100);
at least one temperature sensor (25) to monitor the user's temperature or the ambient temperature;
at least one distance sensor (26) to measure the distance between the user (A) and the phototherapy device (200), and
at least one light sensor (28) to detect the wavelength and the intensity of light received by the eyewear (100).

3. The eyewear (100) as claimed in claim 2, wherein the temperature sensor (25) is adapted to measure the ambient temperature around the user (A) or the temperature of the user (A), and based on the measured temperature, the eyewear (100) adjusts the intensity of the light of the phototherapy device (200) to ensure user (A) comfort and safety.

4. The eyewear (100) as claimed in claim 1, wherein the plurality of sensors (20) comprises:
at least one biometric sensor (24) for measuring biometric data relating to the skin and/or eyes of a user (A) to confirm that the eyewear (100) is properly positioned over the user's eyes to determine a wearing status of the eyewear (100);
at least one temperature sensor (25) to monitor the user's temperature;
at least one distance sensor (26) to measure the distance between the user (A) and the phototherapy device (200), and
at least one light sensor (28) to detect the wavelength and the intensity of light received by the eyewear (100).

5. The eyewear (100) as claimed in claim 4, wherein the biometric sensor (24) is configured to confirm that the eyewear (100) is properly positioned over the user's eyes, which in turn activates the phototherapy device (200) by communicating the wearing status with the phototherapy device (200) through the communication unit (40).

6. The eyewear (100) as claimed in claim 1, wherein the processing unit (30) is adapted to analyze a wearing status of the eyewear (100) based on the data received from the plurality of sensors (20), wherein if the processing unit (30) confirms the eyewear (100) is worn by the user (A), the communication unit (40) communicates the wearing status with the phototherapy device (200) to activate the phototherapy device (200).

7. The eyewear (100) as claimed in claim 1, wherein the processing unit (30) is configured to analyze the intensity of light coming from the phototherapy device (200), and the distance between the user (A) and the phototherapy device (200) to transmit the alert based on the analyzed light intensity and the distance between the user and the phototherapy device (200) to the user (A) using a speaker (29) arranged within the frame (10), wherein, the alert is a voice command to maintain the optimal distance between the user (A) and the phototherapy device (200).

8. The eyewear (100) as claimed in claim 1, wherein the processing unit (30) is configured to analyze ambient environmental conditions such as the light intensity and the wavelength detected by a light sensor (28) of the plurality of sensors (20) and adjust the light output of the phototherapy device (200) to compensate for changes in external light conditions.

9. The eyewear (100) as claimed in claim 1, wherein the operational parameters of the phototherapy device (200) include:
an activation or deactivation of the phototherapy device (200);
an intensity of the light emitted by the phototherapy device (200);
a duration of light exposure;
a pulsation rate of the emitted light;
a color spectrum of the emitted light;
an angular distribution of the emitted light to optimize therapeutic effectiveness;
timing of light emission sequences to match circadian rhythms or treatment protocols;
a modulation of light based on real-time environmental feedback through the plurality of sensors (20);
adaptive control of the light emission based on the user's physiological data, such as stress levels or heart rate;
a synchronization of light therapy with other phototherapy devices that are in use by the user (A).

10. The eyewear (100) as claimed in claim 1, wherein the processing unit (30) continuously monitors data from the plurality of sensors (20) arranged in the eyewear (100) to provide data about whether the eyewear (100) is properly positioned on the user's head and aligned over the user's eyes during the phototherapy treatment to control at least one operational parameter of the phototherapy device (200).

11. The eyewear (100) as claimed in claim 1, wherein the eyewear (100) includes an energy storage unit (90) arranged within the frame (10), which provides energy to the processing unit (30), the communication unit (40), and the plurality of sensors (20), enabling the operation of the eyewear (100), wherein the energy storage unit (90) is a rechargeable battery.

12. The eyewear (100) as claimed in claim 1, wherein the communication unit (40) includes a first body-communication unit (42) that is adapted to communicate a wearing status with the phototherapy device (200) through physical contact of the user (A), wherein, the phototherapy device (200) includes a second body-communication unit (242) adapted to receive the wearing status from the first body-communication unit (42) of the eyewear (100) to turn ON the phototherapy device (200).

13. The eyewear (100) as claimed in claim 12, wherein the first body-communication unit (42) and the second body-communication unit (242) are adapted to transmit the wearing status through the body of the user (A) to activate the phototherapy device (200) that is a handheld device (200′) holdable within the hands of the user (A).

14. The eyewear (100) as claimed in claim 12, wherein the second body-communication unit (242) detects the wearing status transmitted from the first body-communication unit (42), ensuring that the phototherapy device (200) only activates when the user (A) is wearing the eyewear (100) and is in the correct position to receive the phototherapy treatment.

15. The eyewear (100) as claimed in claim 12, wherein the first body-communication unit (42) and the second body-communication unit (242) communicate with each other through body-communication technology, which uses the conductive properties of a human body to transmit signals between the eyewear (100) and the phototherapy device (200).

16. An eyewear (100) to be used during phototherapy, the eyewear (100) comprising:
a frame (10) equipped with a pair of lenses (11a, 11b);
a plurality of sensors (20) arranged in the frame (10), the plurality of sensors (20) configured to detect parameters including at least one biometric data of the user (A);
a processing unit (30) communicatively coupled with the plurality of sensors (20), the processing unit (30) is adapted to process a data received from the plurality of sensors (20), and
a communication unit (40) interfaced with the processing unit (30) to receive a processed data and is adapted to interact with a phototherapy device (200) to control at least one operational parameter of the phototherapy device (200) based on the processed data;
wherein, the plurality of sensors (20) includes a biometric sensor (24) to detect the biometric data of the user (A) for confirming the correct positioning of the eyewear (100) over the user's eyes, and upon such confirmation, the communication unit (40) sends a wearing status to activate the phototherapy device (200).

17. The eyewear (100) as claimed in claim 16, wherein the biometric sensor (24) is adapted to detect the presence of the user's eyes, wherein the detection of the presence of the user's eyes verifies that the eyewear (100) is worn on the eyes and not on any other part of the body.

18. An eyewear (100) to be used during phototherapy, the eyewear (100) comprising:
a frame (10) equipped with a pair of lenses (11a, 11b);
a plurality of sensors (20) arranged in the frame (10) for detecting parameters;
a processing unit (30) communicatively coupled with the plurality of sensors (20), the processing unit (30) is adapted to process data received from the plurality of sensors (20), and
a communication unit (40) arranged in the frame (10), wherein the communication unit (40) interfaces with the processing unit (30) to receive a processed data and is adapted to interact with a phototherapy device (200) to control at least one operational parameter of the phototherapy device (200) based on the processed data and/or the communication unit (40) is adapted to transmit an alert to a user (A) based on the processed data;
wherein the communication unit (40) includes a first body-communication unit (42) adapted to transmit a wearing status to the phototherapy device (200) via physical contact with the user (A);
wherein, the phototherapy device (200) includes a second body-communication unit (242) adapted to receive the wearing status from the first body-communication unit (42) and, to activate the phototherapy device (200) based on the received wearing status.

19. The eyewear (100) as claimed in claim 18, wherein the eyewear (100) is adapted to communicate with the phototherapy device (200) which is a handheld phototherapy device (200′) configured to receive the wearing status from the first body-communication unit (42) via physical contact with the user (A), wherein the first body-communication unit (42) communicates the wearing status continuously to keep the handheld phototherapy device (200) ON until the handheld phototherapy device (200″) is held by the user (A) and the eyewear (100) is properly positioned over the user's eyes.

20. The eyewear (100) as claimed in claim 18, wherein the first body-communication unit (42) and the second body-communication unit (242) communicate with each other through body-communication technology, which uses the conductive properties of the human body to transmit signals between the eyewear (100) and the phototherapy device (200).
